(19) United States
(12) Patent Application Publication
(10) Pub. No.: US 2011/0201579 A1
(43) Pub. Date: Aug. 18, 2011

(54) Method of Improving the Overall Health of a Patient by Application of Pregnenolone

(76) Inventor: John R. Woodward, Dallas, TX (US)

(21) Appl. No.: 12/856,227

(22) Filed: Aug. 13, 2010

Related U.S. Application Data
(60) Provisional application No. 61/305,594, filed on Feb. 18, 2010.

(51) Int. Cl. A61K 31/56; A61P 25/28; A61P 25/24; A61P 9/12; A61P 9/00; A61P 5/24; A61P 3/04

(52) U.S. Cl. 514/170

(57) ABSTRACT

A method is described herein for improving the overall health of a patient by providing subcutaneous pellets containing pregnenolone to at least one part of the body of the patient. In another embodiment, pregnenolone is mixed with DHEA into a cream and the resulting pregnenolone/DHEA cream is applied to at least one part of a body.

### BRIEF DESCRIPTION OF THE DRAWINGS

For a more complete understanding of the present invention, including its features and advantages, reference is now made to the detailed description of the invention taken in conjunction with the accompanying drawing in which:

FIG. 1 is a diagram utilized to explain an embodiment of the present invention.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

Pregnenolone is a natural hormone that is sometimes referred to as the body's “master hormone” since it is the precursor for all other steroid hormones. It is converted directly into dehydroepiandrosterone (DHEA) and/or progesterone. DHEA converts to testosterone and estrogens; progesterone converts to estrogens, cortisol and aldosterone. It is this succession of conversions that makes human life possible. Without pregnenolone, there can be no human steroid hormone production.

Back in the 1940's, when researchers started experimenting with the use of pregnenolone, they realized that it could be helpful for people under stress and it could increase energy in those who were fatigued. However, about the same time, cortisol was discovered.

Cortisol stole the limelight. When cortisol was given to individuals with rheumatoid arthritis, they experienced outstanding short-term improvements. Photographs of these remarkable recoveries were circulated and the medical community was impressed. Scientists then basically put pregnenolone aside to focus on cortisol. The structure of cortisol was altered to make similar molecules such as dexamethasone and prednisone, much more powerful steroids. Dexamethasone and other similar corticosteroids could be patented, and thus a pharmaceutical company could make a lot of money. Pregnenolone has stayed in relative obscurity since the 1940's, with only rare mentions in the medical literature. However, there have been few studies published on pregnenolone in recent years, and only a couple of the studies involve human subjects.

Some people find pregnenolone improves energy, vision, memory, clarity of thinking, wellbeing, and often sexual enjoyment or libido. Pregnenolone may be considered a good brain enhancer in those who are deficient. Studies in rodents show pregnenolone to be one of the most effective and powerful memory boosters. In addition, pregnenolone may increase levels of acetylcholine in the hippocampus and other memory regions in the brain. Some women report lessening of hot flashes or premenstrual symptoms.

Pregnenolone production has also been found to decrease as humans get older. Like many health-promoting hormones, levels of pregnenolone drop with age. Although the data is not as abundant or definitive for pregnenolone as it is for DHEA, Dr. Eugene Roberts, a pioneer in hormone research, believes that the age-related drop in pregnenolone is as dramatic as the drop in DHEA. At 75, our bodies typically make 60% less pregnenolone than at age 35. This is a point of great concern, considering pregnenolone's numerous protective, health-promoting properties Pregnenolone replacement therapy normally consists of patients taking oral supplements but the therapy is still evolving.

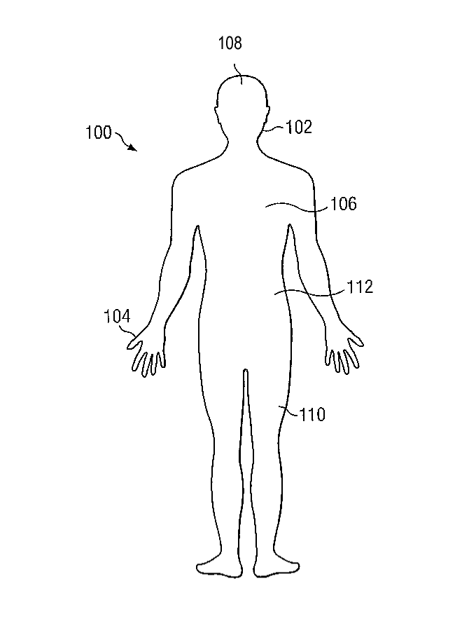

An embodiment of the present invention utilizes pellets of pregnenolone that are injected into the subcutaneous fat (fat beneath the skin) of a patient at various areas. FIG. 1 illustrates an example patient 100. In this embodiment, the pregnenolone pellets are injected into the subcutaneous fat in the hands 104 of the patient 100. Moreover, the pellets can also be injected into the thighs 110 of the patient. This embodiment contemplates an injection every 10-12 weeks in each of those areas. In addition, the pellets used are 25 and 50 milligram. First, the 25 milligram pellets are used, then the blood pressure of the patient should be checked before use of the 50 milligram pellets. If the blood pressure rises, use of the 50 milligram pellets should be avoided. Applications of this type have shown to positively effect the energy level of patients as well lowering the stress levels of the patients.

Another embodiment includes equal parts of pregnenolone and DHEA combined into a cream and is applied directly on the skin. Application directly on the skin helps replenish the pregnenolone and DHEA normally produced within the skin. Consequently, direct skin application has been found to improve the overall health of a patient. In addition, positive effects have been found if the cream is applied to thin vascular skin such as between the inner arm and the side of the arm or flank.

In this embodiment, the pregnenolone/DHEA cream is applied topically to skin on the face 102, hands 104 and the chest 106 of the patient 100. This embodiment contemplates two applications per day in each of those areas. Applications of this type have shown to positively effect the mood and well-being of patients. In addition, these types of applications have also shown to decrease pro-inflammatory cytokine secretion production stimulated by ACTH and stress. Such, the applications have been shown to improve the muscle to fat ratio of patients.

The pregnenolone/DHEA cream can also be applied to the nipples and clitoris of a female patient and the head of a male's penis. This application has been shown to improve libido and sexual response of the patients. One reason that this type of application helps is because the combined pregnenolone/DHEA cream has been shown to have a strong affinity for the Sex Hormone Binding Globulin (SHBG).

These types of applications have a strong affinity for serum albumin and positively effect cognition, depression and mobility. In addition, the applications affect global self-rated health for men and especially women. Moreover, the applications lower blood pressure and improve cardiovascular health in men. Further, the applications are antagonistic to cortisol and thus decrease abdominal fat. The applications also improve the skin and joints and increase hair growth.

Although this invention has been described with reference to an illustrative embodiment, this description is not intended to limit the scope of the invention. Various modifications and combinations of the illustrative embodiments as well as other embodiments of the invention will be apparent to persons skilled in the art upon reference to the description. It is therefore intended that the appended claims accomplish any such modifications or embodiments.

We claim:

1. A method of applying a mixture of pregnenolone and DHEA topically to the skin of a patient.

2. The method of claim 1 wherein the mixture is ½ pregnenolone and ½ DHEA

3. The method of claim 1 wherein the mixture is applied to a thigh portion of the patient.

4. The method of claim 1 wherein the mixture is applied to a face portion of the patient.

5. The method of claim 1 wherein the mixture is applied to a chest portion of the patient.

6. The method of claim 1 wherein the mixture is applied to a sexual organ of the patient.

7. A method of injecting pregnenolone pellets into the subcutaneous fat of a patient.

8. The method of claim 7 wherein the pellets are 25 milligrams.

9. The method of claim 8 further including checking a blood pressure of the patient.

10. The method of claim 9 wherein the pellets are increased to 50milligrams.
